(19) United States
(12) Patent Application Publication
(10) Pub. No.: US 2019/0046681 A1
(43) Pub. Date: Feb. 14, 2019

(54) System and Method for Preserving and Delivering a Therapeutic Gas to a Wound

(71) Applicant: Avent, Inc., Alpharetta, GA (US)

(72) Inventors: Ryan A. Squires, Tigard, OR (US); John P. Gann, Portland, OR (US); Jennifer L. Lauder, Beaverton, OR (US); David C. Blessing, Portland, OR (US)

(21) Appl. No.: 16/076,746

(22) PCT Filed: Feb. 10, 2017
(86) PCT No.: PCT/US2017/017286

§ 371 Date: Aug. 9, 2018

Related U.S. Application Data
(60) Provisional application No. 62/295,638, filed on Feb. 16, 2016.

(51) Int. Cl. A61L 15/42; A61L 15/24; A61L 15/28; A61L 15/44; A61M 35/00; A61L 15/60

(52) U.S. Cl. CPC A61L 15/425; A61L 15/24; A61L 15/28; A61L 15/44; A61M 35/00; A61M 2205/0205; A61M 2202/0208; A61M 2202/0266; A61M 2202/0275; A61M 2202/0225; A61M 2202/0283; A61L 15/60

(57) ABSTRACT

A system that includes a biocompatible polymeric matrix and a separately-stored fluid for improving delivery of a therapeutic gas to an area beneath a surface of intact skin or a wound site is provided. The system can preserve the therapeutic gas in the matrix such that its concentration remains generally constant until the system is ready for use, at which time the separately-stored fluid can be added or exposed to the matrix to activate the matrix and thus increase the permeability of the biocompatible polymeric matrix to the therapeutic gas, thus facilitating delivery of the therapeutic gas for the purpose of the treatment of wounds and intact skin is provided. A method of using the, as well as a deactivated matrix and its method of use for delivery of a therapeutic gas, are also provided.

### RELATED APPLICATIONS

The present application claims priority to U.S. Provisional Application Ser. No. 62/295,638, filed on Feb. 16, 2016, which is incorporated herein in its entirety by reference thereto.

### FIELD OF THE INVENTION

This invention generally relates to wound care, and more particularly to the controlled preservation and delivery or release of a therapeutic gas (e.g., oxygen) from a biocompatible polymeric matrix to a wound or surface of intact skin.

### BACKGROUND OF THE INVENTION

Wound healing involves the stages of inflammation, proliferation, and maturation. Oxygen plays an important role in the inflammation and proliferation stages, and oxygen consumption is increased during these stages. As such, a condition known as hypoxia, in which a region of the body is deprived of adequate oxygen supply, can impede the wound healing process in both acute and chronic wounds. Thus, clinicians and medical device companies have attempted various ways to deliver topical oxygen to a wound. One challenge in using oxygen as a topical active agent is its tendency to be evanescent due to its gaseous form.

To circumvent this issue, a plastic covering can be secured over a wound site, and the space between the covering and the wound site can be filled with oxygen gas using, for example, a portable oxygen generator. However, such a system is often cumbersome to administer and awkward for the patient. Another option is to expose patients to pure oxygen in a pressurized hyperbaric chamber. This increases the level of oxygen carried within the blood which, in turn, oxygenates a wound site from the capillary bed. While treatment of chronic wounds using hyperbaric oxygen has been effective, it has its drawbacks. For example, treatment with hyperbaric oxygen is expensive, requires a trained staff, and requires a patient to travel to a medical facility having a hyperbaric chamber. In addition, systemic exposure to pure oxygen can increase a patient's risk for oxygen poisoning. Alternatively, a primary wound dressing containing oxygen gas can be placed over a wound site to deliver oxygen to the wound site. However, the oxygen must be dissolved to be utilized by cells during the various stages of wound healing, and the oxygen gas must consequently cross multiple boundaries to become biologically available to the cells. As a result, only a fraction of the dissolved oxygen reaches the cells for utilization during the wound healing process.

In light of the above, a need exists for an improved therapeutic gas delivery system and method of treating a wound site or area of intact skin using the delivery system, where the release of a therapeutic gas such as oxygen can be controlled and optimized in an easy-to-use, convenient system.

### SUMMARY OF THE INVENTION

In accordance with one embodiment of the present invention, a therapeutic gas preservation and delivery system is provided. The system includes a biocompatible polymeric matrix including a polymer and closed cells, wherein a generally constant concentration of a therapeutic gas is stored within biocompatible polymeric matrix, further wherein the biocompatible polymeric matrix includes a surface configured to directly contact a surface of intact skin or a wound site; and a separately-stored fluid. Maintaining separation between the biocompatible polymeric matrix and the separately-stored fluid preserves the concentration of the therapeutic gas stored in the biocompatible polymeric matrix, and adding or exposing the separately-stored fluid to the biocompatible polymeric matrix facilitates delivery of the therapeutic gas to an area beneath the surface of intact skin or the wound site, where the therapeutic gas is dissolved in the separately-stored fluid that has been added or exposed to the biocompatible polymeric matrix.

In one particular embodiment, the biocompatible polymeric matrix can be exposed to the separately-stored fluid for a time period ranging from about 5 seconds to about 30 minutes.

In another embodiment, adding or exposing the separately-stored fluid to the biocompatible polymeric matrix can result in an on-demand therapeutic gas delivery rate ranging from about 1 milliliter/(m²·hour) to about 10,000 milliliters/(m²·hour).

In still another embodiment, the separately-stored fluid can include water, saline, a wetting agent, chemically simulated wound fluid, wound exudate, or a combination thereof.

In yet another embodiment, the therapeutic gas can be dissolved in the separately-stored fluid after the separately-stored fluid is added or exposed to the biocompatible polymeric matrix such that the therapeutic gas moves along a diffusion gradient for delivery beneath the surface of intact skin or the wound site.

In an additional embodiment, the therapeutic gas can include oxygen, nitrogen, nitric oxide, carbon dioxide, air, nitrous oxide, hydrogen sulfide, a gaseous prostaglandin, a flurane derivative, or a combination thereof.

In one more embodiment, the therapeutic gas can be transported through the surface of intact skin or transported to the wound site for up to about 10 days.

In a further embodiment, the biocompatible polymeric matrix can be capable of absorbing at least five times its weight in fluid.

In one particular embodiment, the biocompatible polymeric matrix can be generally free of moisture prior to addition of the separately-stored fluid.

In another embodiment, the biocompatible polymeric matrix can exhibit a low permeability or impermeability to the therapeutic gas prior to addition of the separately-stored fluid and can exhibit an increased permeability to the therapeutic gas after addition of the separately-stored fluid.

In still another embodiment, the biocompatible polymeric matrix can be hydrophilic.

In yet another embodiment, the polymer can be at least partially cross-linked.

In an additional embodiment, the polymer can be an absorbent, elastomeric, and biocompatible polymer or copolymer. For instance, the absorbent, elastomeric, and biocompatible polymer or copolymer can include polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof. In addition, the biocompatible polymeric matrix can further include a polysaccharide.

In one more embodiment, the biocompatible polymeric matrix can include an active agent. Further, adding or exposing the separately-stored fluid to the biocompatible polymeric matrix facilitates delivery of the active agent to an area beneath the surface of intact skin or the wound site, wherein the active agent is dissolved in the separately-stored fluid that has been added or exposed to the biocompatible polymeric matrix.

In a further embodiment, the biocompatible polymeric matrix can include a superabsorbent material.

In one particular embodiment, the system can include one or more layers of the biocompatible polymeric matrix.

In another embodiment, the system can include an occlusive backing layer disposed on a surface of the biocompatible polymeric matrix, wherein the occlusive backing layer is exposed to the ambient environment.

In still another embodiment, the biocompatible polymeric matrix can further include open cells.

In accordance with an additional embodiment of the present invention, a method for preserving and delivering a therapeutic gas to an area of intact skin or a wound site is provided. The method includes providing a system that includes a biocompatible polymeric matrix comprising a polymer and closed cells, wherein a generally constant concentration of a therapeutic gas is stored within the biocompatible polymeric matrix, and a separately-stored fluid, where maintaining separation between the biocompatible polymeric matrix and the separately-stored fluid preserves the concentration of the therapeutic gas stored in the biocompatible polymeric matrix; adding or exposing the separately-stored fluid to the biocompatible polymeric matrix, wherein the therapeutic gas is dissolved in the separately-stored fluid after the separately-stored fluid is added or exposed to the biocompatible polymeric matrix; and applying the biocompatible polymeric matrix to a surface of intact skin or a wound site, wherein the therapeutic gas is delivered an area beneath the surface of intact skin or the wound site.

In one particular embodiment, the biocompatible polymeric matrix can be exposed to the separately-stored fluid for a time period ranging from about 5 seconds to about 30 minutes prior to applying the biocompatible polymeric matrix to the surface of intact skin or the wound site.

In another embodiment, adding or exposing the separately-stored fluid to the biocompatible polymeric matrix can result in an on-demand therapeutic gas delivery rate ranging from about 1 milliliter/(m²·hour) to about 10,000 milliliters/(m²·hour).

In still another embodiment, the separately-stored fluid can be applied to the biocompatible polymeric matrix while the biocompatible polymeric matrix is applied to the surface of intact skin or the wound site.

In yet another embodiment, the separately-stored fluid can be applied to a skin-contacting surface of the biocompatible polymeric matrix.

In an additional embodiment, the separately-stored fluid can be applied to the biocompatible polymeric matrix by soaking, dipping, coating, or spraying.

In one more embodiment, the separately-stored fluid can include water, saline, a wetting agent, chemically simulated wound fluid, wound exudate, or a combination thereof.

In a further embodiment, the therapeutic gas can be dissolved in the separately-stored fluid after the separately-stored fluid is added or exposed to the biocompatible polymeric matrix such that the therapeutic gas moves along a diffusion gradient for delivery beneath the surface of intact skin or the wound site.

In one particular embodiment, the therapeutic gas can include oxygen, nitrogen, nitric oxide, carbon dioxide, air, nitrous oxide, hydrogen sulfide, a gaseous prostaglandin, a flurane derivative, or a combination thereof.

In another embodiment, the therapeutic gas can be transported through the surface of intact skin or transported to the wound site for up to about 10 days.

In still another embodiment, the biocompatible polymeric matrix can absorb at least five times its weight in fluid.

In yet another embodiment, the biocompatible polymeric matrix can be generally free of moisture prior to addition of the separately-stored fluid.

In an additional embodiment, the biocompatible polymeric matrix can be dried or desiccated prior to the addition of the separately-stored fluid.

In one more embodiment, the biocompatible polymeric matrix can exhibit low permeability or no permeability to the therapeutic gas prior to addition of the separately-stored fluid and can exhibit an increased permeability to the therapeutic gas after addition of the separately-stored fluid.

In a further embodiment, the polymer can be hydrophilic and at least partially cross-linked. In addition, the polymer can be an absorbent, elastomeric, and biocompatible polymer or copolymer such as polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof. Further, the biocompatible polymeric matrix can also include a polysaccharide or a superabsorbent material.

In one more embodiment, the biocompatible polymeric matrix can also include an active agent. Further, adding or exposing the separately-stored fluid to the biocompatible polymeric matrix can facilitate delivery of the active agent to an area beneath the surface of intact skin or the wound site, wherein the active agent is dissolved in the separately-stored fluid that has been added or exposed to the biocompatible polymeric matrix.

In one particular embodiment, the biocompatible polymeric matrix can include one or more layers of the biocompatible polymeric matrix.

In another embodiment, the biocompatible polymeric matrix can include open cells.

In accordance with one more embodiment of the present invention, a biocompatible polymeric matrix for the delivery of a therapeutic gas to an area beneath a surface of intact skin or a wound site is provided. The biocompatible polymeric matrix includes a polymer and closed cells, where a generally constant concentration of a therapeutic gas is contained within the closed cells, where the biocompatible polymeric matrix exists in a deactivated state so that the biocompatible polymeric matrix exhibits a low permeability or impermeability to the therapeutic gas, and whereby the biocompatible polymeric matrix preserves the concentration of therapeutic gas contained within the closed cells.

In one particular embodiment, the biocompatible polymeric matrix can be deactivated by drying or desiccation.

In another embodiment, the biocompatible polymeric matrix can be configured to be activated when contacted with a fluid or ambient moisture, wherein upon activation, the biocompatible polymeric matrix exhibits an increased permeability to the therapeutic gas.

In still another embodiment, upon activation, the biocompatible polymeric matrix can deliver the therapeutic gas in dissolved from to the area beneath the surface of intact skin or the wound site at a rate ranging from about 1 milliliter/(m²·hour) to about 10,000 milliliters/(m²·hour).

In yet another embodiment, the therapeutic gas can include oxygen, nitrogen, nitric oxide, carbon dioxide, air, nitrous oxide, hydrogen sulfide, a gaseous prostaglandin, a flurane derivative, or a combination thereof.

In an additional embodiment, the therapeutic gas can be transported through the surface of intact skin or transported to the wound site for up to about 10 days once the biocompatible polymeric matrix is activated.

In one more embodiment, the biocompatible polymeric matrix can be capable of absorbing at least five times its weight in fluid.

In a further embodiment, the polymer can be hydrophilic and can be at least partially cross-linked.

In one particular embodiment, the polymer can be an absorbent, elastomeric, and biocompatible polymer or copolymer. For example, the absorbent, elastomeric, and biocompatible polymer or copolymer can include polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof. Further, the biocompatible polymeric matrix can also include a polysaccharide.

In another embodiment, the biocompatible polymeric matrix can also include an active agent. Further, the biocompatible polymeric matrix can be configured to be activated when contacted with a fluid or ambient moisture, wherein upon activation, the biocompatible polymeric matrix exhibits an increased permeability to the active agent.

In still another embodiment, the biocompatible polymeric matrix can include a superabsorbent material.

In yet another embodiment, the matrix can include one or more layers.

In an additional embodiment, an occlusive backing layer is disposed on a surface of the biocompatible polymeric matrix, wherein the occlusive backing layer is exposed to the ambient environment.

In one more embodiment, the biocompatible polymeric matrix can further include open cells.

A method of enhancing delivery of the therapeutic gas from the biocompatible polymeric matrix to the area beneath the surface of intact skin or the wound site is also contemplated by the present invention. The method includes exposing the biocompatible polymeric matrix to a fluid or ambient moisture.

In one particular embodiment, exposure to the fluid or ambient moisture can increase the permeability of the biocompatible polymeric matrix to the therapeutic gas.

In another embodiment, the biocompatible polymeric matrix can be exposed to the fluid or ambient moisture for a time period ranging from about 5 seconds to about 30 minutes prior to applying the biocompatible polymeric matrix to the surface of intact skin or the wound site.

In still another embodiment, the biocompatible polymeric matrix can be exposed to the fluid or ambient moisture while the biocompatible polymeric matrix is in contact with the surface of intact skin or the wound site.

In an additional embodiment, the biocompatible polymeric matrix can also include an active agent. Further, exposure to the fluid or ambient moisture increases the permeability of the biocompatible polymeric matrix to the active agent.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE FIGURES

A full and enabling disclosure of the present invention, including the best mode thereof to one skilled in the art, is set forth more particularly in the remainder of the specification, including reference to the accompany figures, in which:

FIG. 1 illustrates a cross-sectional view of an absorbent, biocompatible polymeric matrix component of a therapeutic gas preservation and delivery system that is contemplated by the present invention;

FIG. 2 illustrates a perspective view of one embodiment of a therapeutic gas preservation and delivery system that includes the matrix of FIG. 1;

FIG. 3 illustrates a perspective view of another embodiment of a therapeutic gas preservation and delivery system contemplated by the present invention that includes the matrix of FIG. 1 as well as an occlusive backing;

FIG. 4 is a graph illustrating the ability of the biocompatible polymeric matrix of the present invention to provide moisture donation to a wound site or surface of intact skin after being soaked in water for a time period ranging from 1 minute to 30 minutes;

FIG. 5 is a graph illustrating the levels of total dissolved oxygen (TDO) that can penetrate a surface of intact skin and diffuse to the dermal capillary bed within one minute after placing the biocompatible polymeric matrix of the present invention on the surface of intact skin when the biocompatible polymeric matrix of the present invention is soaked in water for five seconds, one minute, and five minutes prior to placing the matrix on the surface of intact skin, as normalized against a control in which the matrix was not soaked in water prior to placing the matrix on the surface of intact skin;

FIG. 6 is a graph illustrating the levels of total dissolved oxygen (TDO) that can penetrate a surface of intact skin and diffuse to the dermal capillary bed within one hour, three hours, and six hours after placing the biocompatible polymeric matrix of the present invention on the surface of intact skin when the matrix is soaked in water for three minutes prior to placing the matrix on the surface of intact skin, as normalized against a control in which the matrix was not soaked in water prior to placing the matrix on the surface of intact skin;

FIG. 7 is a graph illustrating the levels of total dissolved oxygen (TDO) that can penetrate a surface of intact skin and diffuse to the dermal capillary bed within one hour, three hours, six hours, twelve hours, eighteen hours, and twenty-four hours after placing the biocompatible polymeric matrix of the present invention on the surface of intact skin when the matrix is soaked in water for five minutes prior to placing the matrix on the surface of intact skin, as normalized against a control in which the matrix was not soaked in water prior to placing the matrix on the surface of intact skin;

FIG. 8 is a graph illustrating the levels of total dissolved oxygen (TDO) that can penetrate a surface of intact skin and diffuse to the dermal capillary bed within one hour, three hours, and six hours after placing the biocompatible polymeric matrix of the present invention on the surface of intact skin when the matrix is soaked in water for fifteen minutes prior to placing the matrix on the surface of intact skin, as normalized against a control in which the matrix was not soaked in water prior to placing the matrix on the surface of intact skin;

FIG. 9 is a graph illustrating the ability of the biocompatible polymeric matrix of the present invention to absorb saline;

FIG. 10 is a graph comparing the amount of nitrogen gas in the biocompatible polymeric matrix of the present invention with the amount of nitrogen gas in a dry environment (pouch), showing that the gas composition in the matrix material is independent of the gas composition in a dry environment; and

FIG. 11 is a graph illustrating the change in oxygen content remaining in the biocompatible polymeric matrix of the present invention as the moisture content of the matrix is increased.

Repeat use of reference characters in the present specification and drawings is intended to represent the same or analogous features or elements of the present invention.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a therapeutic gas preservation and delivery system and method for applying such a system to a surface of intact skin or to a wound site, as well as a biocompatible polymeric matrix that can be used in the system. It is to be understood that the term “wound site” can refer to an area associated with a cut, an abrasion, a cavity, an ulcer, a tunnel, a puncture wound, a wound resulting from a Mohs procedure or radiation treatment, or any other wound (gunshot wound, stab wound, etc.) and that the biocompatible polymeric matrix is capable of conforming to fill in the void space at the wound site. Such wound sites may have dimensions (length, width, and/or depth) spanning up to 1.5 inches.

In one particular embodiment, the system includes a biocompatible polymeric matrix and a separately-stored fluid. The biocompatible matrix, which includes a surface configured to directly contact a surface of intact skin or a wound site, also includes a polymer and closed cells, where a generally constant concentration of a therapeutic gas is stored within biocompatible polymeric matrix. Further, the matrix and fluid are stored separately, as the present inventors have found that maintaining separation between the biocompatible polymeric matrix and the separately-stored fluid preserves the concentration of the therapeutic gas stored in the biocompatible polymeric matrix. Meanwhile, the present inventors have found that adding or exposing the separately-stored fluid to the biocompatible polymeric matrix activates the biocompatible polymeric matrix and facilitates delivery of the therapeutic gas to an area beneath the surface of intact skin or the wound site, where the therapeutic gas is dissolved in the separately-stored fluid that has been added or exposed to the biocompatible polymeric matrix.

The present invention also contemplates a method for preserving and delivering a therapeutic gas to an area of intact skin or a wound site. The method includes adding a separately-stored fluid to a biocompatible polymeric matrix comprising a polymer and closed cells, where a generally constant concentration of a therapeutic gas is stored within the biocompatible polymeric matrix. By maintaining separation between the biocompatible polymeric matrix and the separately-stored fluid, the concentration of the therapeutic gas stored in the biocompatible polymeric matrix is preserved. Then, once the separately-stored fluid is introduced (e.g., added or exposed) to the biocompatible polymeric matrix, the therapeutic gas stored in the matrix becomes dissolved in the separately-stored fluid, after which the biocompatible polymeric matrix is applied to a surface of intact skin or a wound site, and the therapeutic gas is delivered to an area beneath the surface of intact skin or the wound site.

A biocompatible polymeric matrix for the delivery of a therapeutic gas to an area beneath a surface of intact skin or a wound site is also contemplated by the present invention. The biocompatible polymeric matrix comprises a polymer and closed cells, and a generally constant concentration of a therapeutic gas is contained within the closed cells. Further, the biocompatible polymeric matrix is deactivated so that the biocompatible polymeric matrix exhibits decreased permeability to the therapeutic gas, whereby the present inventors have found that the biocompatible polymeric matrix preserves the concentration of therapeutic gas contained within the closed cells due to its deactivated state. This enables the delivery of the maximum amount of therapeutic gas once the matrix is activated by increasing its permeability via the addition of a fluid or ambient moisture (e.g., moisture from the ambient environment, air, or skin).

The present inventors have found that the aforementioned system and biocompatible polymeric matrix facilitates the delivery of a therapeutic gas to a wound site or surface of intact skin in an efficient manner, where less of the therapeutic gas is lost during storage of the product due, for instance, to the decreased permeability of the matrix when it is in a deactivated state. Then, by supplying a fluid to the matrix to reactivate the matrix and increase its permeability, the therapeutic gas can be delivered to the desired location in an on-demand fashion, wherein increasing the amount of time that the matrix is contacted with the fluid can increase the amount of therapeutic gas that is delivered and/or the rate at which the therapeutic gas is delivered. The various components of the matrix and system, along with their method of use, are discussed in more detail below.

The therapeutic gas preservation and delivery system includes a hydrophilic, biocompatible polymeric matrix that may have a planar or non-planar shape and includes a polymer, where the matrix includes closed cells and walls, although it is also to be understood that the cells can be a combination of closed cells and open cells. In some embodiments, the matrix is comprised of substantially closed cells. In other embodiments, the matrix contains from about 85% to about 100% closed cells, such as from about 90% to about 99.9% closed cells, such as from about 99% to about 99.5% closed cells.

The therapeutic gas preservation and delivery system further includes a therapeutic gas (e.g., a gas such as oxygen), where the therapeutic gas can be contained in the closed cells of the biocompatible polymeric matrix. It should be understood that the therapeutic gas is created during the manufacturing of the biocompatible polymeric matrix (i.e., the therapeutic gas is not created when the matrix is placed on the wound site). The therapeutic gas can be produced when, for example, a reactant, such as a peroxide, is reacted with a catalyst that is a component of the matrix, such as a carbonate, resulting in the formation of oxygen gas. Further, the biocompatible polymeric matrix can exhibit decreased permeability, and, in some embodiments, can be generally impermeable, to the therapeutic gas in order to preserve the concentration of the therapeutic gas in the biocompatible matrix (i.e., the concentration of the therapeutic gas in the closed cells of the biocompatible polymeric matrix is generally constant). The concentration of the therapeutic gas can be preserved or maintained at a constant level until such time that the matrix is to be used (e.g., placed on a surface of intact skin or a wound site for the delivery of the therapeutic gas), where the decreased permeability prevents depletion of the therapeutic gas concentration in the system. Then, once it is desired to use the matrix to deliver a therapeutic gas, the system can be activated such that the biocompatible polymeric matrix exhibits increased permeability to the therapeutic gas, such as via the addition of a fluid (e.g., water, saline, chemically simulated wound fluid, wound exudate, a wetting agent, or a combination thereof, etc.), at which time the therapeutic gas becomes dissolved in the fluid contained in the closed cells and walls of the matrix and can be delivered on-demand to an area beneath the surface of intact skin or the wound site via a diffusion gradient. Although not required, the wetting agent can be included to facilitate the penetration of the therapeutic gas and/or active agents through the skin, particularly when the matrix is placed on intact skin as opposed to a wound. One specific example of a wetting agent that can be utilized is dimethyl sulfoxide (DMSO).

In any event, the biocompatible polymeric matrix can be contacted with the fluid for a time period ranging from about 5 seconds to about 30 minutes, such as from about 30 seconds to about 25 minutes, such as from about 1 minute to about 20 minutes, such as from about 3 minutes to about 15 minutes, whereby increasing the contact time between the biocompatible polymeric matrix and the fluid can increase the amount of dissolved therapeutic gas delivered by the fluid to the area beneath the surface of intact skin or the wound site or transported by the fluid through the surface of intact skin or transported to the wound site for up to about 10 days. Depending on the specific configuration of the matrix and the fluid soak times, the delivery of the therapeutic gas can be selectively controlled such that the therapeutic gas can be delivered or transported continuously over a time period of up to about 10 days, such as a time period of up to about 7 days, such as a time period of up to about 5 days. In other words, contacting the biocompatible polymeric matrix with fluid as described above “activates” the matrix so that it is permeable to the therapeutic gas and can deliver the therapeutic gas “on-demand” to the area beneath the surface of intact skin or the wound site at a rate ranging from about 1 milliliter/(m²·hour) to about 10,000 milliliters/(m²·hour), such as from about 1.5 milliliters/(m²·hour) to about 9500 milliliters/(m²·hour), such as from about 2 milliliters/(m²·hour) to about 9250 milliliters/(m²·hour). In contrast, when in its “deactivated” state, the biocompatible polymeric matrix is generally impermeable and delivers zero to very minimal levels of therapeutic gas and has a delivery rate ranging from 0 milliliters/(m²·hour) to about 0.075 milliliters/(m²·hour), such as from about 0.001 milliliters/(m²·hour) to about 0.05 milliliters/(m²·hour), such as from about 0.005 milliliters/(m²·hour) to about 0.025 milliliters/(m²·hour).

Although any therapeutic gas (e.g., oxygen, nitric oxide, carbon dioxide, carbon monoxide, air, nitrous oxide, hydrogen sulfide, a gaseous prostaglandin, a flurane derivative, etc.) is contemplated as a component of the biocompatible polymeric matrix of the present invention, in one particular embodiment, oxygen can be the therapeutic gas that is utilized. As is known in the art, when oxygen is supplied for the purposes of wound healing, it is known to help with some metabolic processes that rely on oxygen during wound healing.

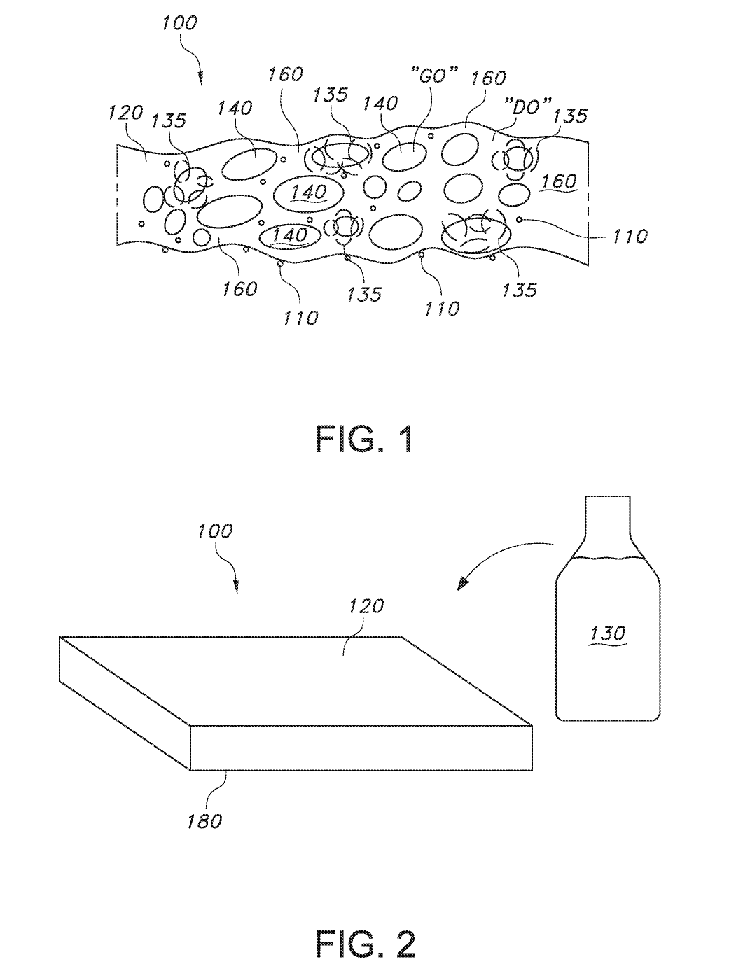

Referring now to FIG. 1, there is illustrated in cross-section of the biocompatible polymeric matrix 120 component of an exemplary therapeutic gas preservation and delivery system 100 (not necessarily to scale). The matrix may include a water-swellable, cross-linked biocompatible polymer network. Exemplary biocompatible matrices and techniques to form the same are disclosed by U.S. Pat. No. 8,679,523 issued Mar. 25, 2014 and U.S. Pat. No. 7,160,553 issued Jan. 9, 2007 to Gibbins, et al., which are incorporated herein by reference.

As mentioned above, the matrix 120 can include closed cells 140 and walls 160. That is, a plurality of gas permeable, elastic, closed cells 140 can be defined by the cross-linked biocompatible polymer network, where the elastic, closed cells are gas permeable when the matrix is activated, as well during formation of the matrix so that the therapeutic gas can be introduced to the closed cells, while the matrix can also be configured such that it exhibits reduced permeability or is generally impermeable when deactivated (e.g., via drying, desiccation, etc.) to preserve the concentration of the therapeutic gas contained therein until it is desired to deliver the therapeutic gas to an area of intact skin or a wound site. Moreover, it is also to be understood that in some embodiments, the matrix 120 can also include a combination of open cells 135 and closed cells 140. In one particular embodiment, the matrix 120 may be a biocompatible polymeric matrix that includes a polymer and an oxygen catalyst and the closed cells 140 may be produced from a reaction between the catalyst and a second reactant (e.g., a peroxide). Deliverable oxygen is contained within the elastic closed cells such that when the matrix 120 is used to treat a wound, oxygen is delivered from the closed cells 140. Generally speaking, gaseous oxygen “GO” can be contained in the closed cells 140 and dissolved oxygen “DO” can be present in moisture in the closed cells 140 and walls 160 (for instance, once fluid is added or exposed to the matrix 120 to activate the matrix as discussed above). However, it is to be understood that the therapeutic gas is not limited to oxygen, and any therapeutic gas can be utilized in the present invention. For instance, therapeutic gases such as nitric oxide, carbon dioxide, and carbon monoxide in gaseous and dissolved form are also contemplated for use in therapeutic gas preservation and delivery system of the present invention. In addition, nitrous oxide, hydrogen sulfide, gaseous prostaglandins (prostacyclin, iloprost, teprostinol, beraprost, etc.) and flurane derivatives are also contemplated for use in the present invention. Specifically, the nitrous oxide and flurane derivatives can provide an analgesic effect, while the nitric oxide, prostaglandins, and hydrogen sulfide can be used for vasodilation. Further, although the therapeutic gas can be formed by reacting any suitable catalyst with any suitable reactant as discussed in more detail below, it is also to be understood that the therapeutic gas can be introduced by mixing it into the biocompatible polymeric matrix. Generally, after the therapeutic gas has been introduced into the matrix during manufacturing, about 50% to about 95% of the total volume of the biocompatible polymeric matrix is attributed to the therapeutic gas.

Regardless of the particular gas used or the method by which the gas is introduced into the hydrophilic, biocompatible polymeric matrix, the hydrophilic, biocompatible polymeric matrix 120 can be formed from a polymer or copolymer that can be cross-linked into a water-swellable polymer network. By making the cross-linked biocompatible polymer network sufficiently water-swellable, the second reactant (e.g., a peroxide) may be absorbed into the cross-linked biocompatible polymer network.

The biocompatible polymeric matrix should be flexible such that it can define elastic, closed cells that are also gas permeable when that matrix is activated, as well during formation of the matrix so that the therapeutic gas can be introduced to the closed cells, although it is to be understood that matrix can be configured such that it exhibits reduced permeability or is generally impermeable when deactivated (e.g., via drying, desiccation, etc.) to preserve the concentration of the therapeutic gas contained therein until it is desired to deliver the therapeutic gas to an area of intact skin or a wound site. A variety of polymers either naturally derived or synthetic may be used to prepare the polymer network. Polymers that are hydrophilic, cross-linkable, and biocompatible are preferred. Exemplary polymers include, but are not limited to, absorbent biocompatible polymers such as polyacrylamide, polyacrylic acid, sodium polyacrylate, polyethylvinyacetate, polyurethane, polyethylene oxide, polyvinyl alcohol, polyvinyl pyrrolidone, or a combination thereof. Other polymers that can be used include polylysine, polyethylene, polybuterate, polyether, silastic, silicone elastomer, rubber, nylon, vinyl, cross-linked dextran, or a combination thereof. The polymer used to form the cross-linked polymer network can be present in an amount ranging from about 0.25 wt. % and 20 wt. %, such as from about 0.5 wt. % to about 15 wt. %, such as from about 1 wt. % to about 10 wt. % based on the total weight of the biocompatible polymeric matrix of the present invention prior to drying. On the other hand, after dehydration, the polymer used to form the cross-linked polymer network can be present in an amount ranging from about 1 wt. % to about 80 wt. %, such as from about 5 wt. % to about 70 wt. %, such as from about 10 wt. % to about 60 wt. % based on the total weight of the biocompatible polymeric matrix of the present invention on a dry-weight basis.

As mentioned above, the polymer network can be suitably lightly crosslinked to render the material substantially water-insoluble. Crosslinking may, for example, be by irradiation or by covalent, ionic, Van der Waals, or hydrogen bonding. Suitable cross-linking agents can include N,N′-methylene-bisacrylamide, bisacrylylycystamine and diallyltartar diamide and can be present in the biocompatible polymeric matrix of the present invention in an amount ranging from about 0.005 wt. % to about 0.5 wt. %, such as from about 0.01 wt. % to about 0.25 wt. %, such as from about 0.025 wt. % to about 0.15 wt. % based on the total weight of the biocompatible polymeric matrix of the present invention prior to drying. After dehydration, the cross-linking agent can be present in an amount ranging from about 0.01 wt. % to about 4 wt. %, such as from about 0.05 wt. % to about 3 wt. %, such as from about 0.1 wt. % to about 2 wt. % based on the total weight of the biocompatible polymeric matrix of the present invention on a dry-weight basis.

The biocompatible polymeric matrix can also include a polysaccharide, which can, in some embodiments, be a non-gellable polysaccharide. One example of a suitable polysaccharide is dextrin. Dextrin is a non-cross-linked carbohydrate intermediate between starch and sugars, with the general structure (C₆H₁₀O₅)ₓ, where x can be 6 or 7. In one particular embodiment, type III dextrin can be utilized. In another embodiment, the polysaccharide can be a cellulose or a cellulose derivative. For instance, methyl cellulose (MC), carboxymethyl cellulose (CMC), hydroxyethylcellulose (HEC), hydroxypropylmethyl cellulose (HPMC), hydroxypropyl cellulose (HPC), ethyl hydroxyethylcellulose (EHEC), or a combination thereof can be used in the present invention. In still another embodiment, a galactomannan macromolecule can be used in the biocompatible polymeric matrix. One example of such a macromolecule is guar gum. Other suitable examples of suitable polysaccharides include lucerne, fenugreek, honey locust bean gum, white clover bean gum, carob locust bean gum, xanthan gum, carrageenan, sodium alginate, chitin, chitosan, etc. The polysaccharide can be present in an amount ranging from about 0.005 wt. % to about 25 wt. %, such as from about 0.05 wt. % to about 10 wt. %, such as from about 0.1 wt. % to about 5 wt. %, based on the total weight of the biocompatible polymeric matrix of the present invention prior to drying. On the other hand, after dehydration, the polysaccharide can be present in an amount ranging from about 0.5 wt. % to about 60 wt. %, such as from about 1 wt. % to about 55 wt. %, such as from about 10 wt. % to about 50 wt. % based on the total weight of the biocompatible polymeric matrix of the present invention on a dry-weight basis.

As discussed above, the biocompatible polymeric matrix of the present invention can include a therapeutic gas such as oxygen that is introduced to the matrix during manufacturing via the combination of an oxygen catalyst (e.g., a first reactant) and a second reactant. The catalyst may be sodium carbonate or other alkali and alkali earth compounds may be used provided they are consistent with the product being biocompatible. In addition, more than one catalyst may be used. For instance, one catalyst may be derived from a group consisting of salts of alkali metals and alkali earth metals and the second catalyst may include, but are not limited to, organic and inorganic chemicals such as cupric chloride, ferric chloride, manganese oxide, sodium iodide and their equivalents. Other catalysts, include, but are not limited to enzymes such as lactoperoxidase and catalase. The catalyst can be present in an amount ranging from about 0.005 wt. % to about 5 wt. %, such as from about 0.01 wt. % to about 2.5 wt. %, such as from about 0.05 wt. % to about 1 wt. % based on the total weight of the biocompatible polymeric matrix of the present invention prior to drying. On the other hand, after dehydration, the catalyst can be present in an amount ranging from about 0.01 wt. % to about 10 wt. %, such as from about 0.05 wt. % to about 7.5 wt. %, such as from about 0.1 wt. % to about 6 wt. % based on the total weight of the biocompatible polymeric matrix of the present invention on a dry-weight basis.

Meanwhile, the second reactant used in the biocompatible polymeric matrix of the present invention can be hydrogen peroxide, ammonium peroxide, urea peroxide, sodium peroxide, or any other peroxy compound provided that such compounds leave no residue that would be inconsistent with biocompatibility and/or bioabsorption. The present invention contemplates use of components that can generate a therapeutic gas within the matrix and that are safe and effective for use.

For example, an acid catalyst can be incorporated in the matrix followed by perfusion of the matrix with a carbonate to generate carbon dioxide gas within the matrix. The second reactant can be added to the biocompatible polymeric matrix of the present invention in an amount sufficient to create foaming of the biocompatible polymeric matrix to form the dissolved and gaseous oxygen. After dehydration, the second reactant can be present in an amount ranging from 0 wt. % to about 6 wt. %, such as from about 0.001 wt. % to about 3 wt. %, such as from about 0.01 wt. % to about 1 wt. % based on the total weight of the biocompatible polymeric matrix of the present invention on a dry-weight basis.

The biocompatible polymeric matrix may further include a plasticizer. The plasticizers may be glycerol/glycerin, water, propylene glycol and/or butanol and combinations thereof may also be used. If glycerol is used, the glycerol can be present in an amount ranging from about 0.25 wt. % to about 25 wt. %, such as from about 0.5 wt. % to about 12 wt. %, such as from about 1 wt. % to about 10 wt. % based on the total weight of the biocompatible polymeric matrix of the present invention prior to drying. On the other hand, after dehydration, the glycerol can be present in an amount ranging from about 1 wt. % to about 80 wt. %, such as from about 5 wt. % to about 70 wt. %, such as from about 10 wt. % to about 60 wt. % based on the total weight of the biocompatible polymeric matrix of the present invention on a dry-weight basis. Meanwhile, water can be present in an amount ranging from about 50 wt. % to about 98 wt. %, such as from about 60 wt. % to about 95 wt. %, such as from about 70 wt. % to about 80 wt. % based on the total weight of the biocompatible polymeric matrix of the present invention prior to drying.

After dehydration or desiccation, water or moisture can be present in an amount ranging from about 0 wt. % to about 9.2 wt. %, such as from about 0.01 wt. % to about 5 wt. %, such as from about 0.1 wt. % to about 2.5 wt. % based on the total weight of the biocompatible polymeric matrix of the present invention on a dry-weight basis. In other embodiments, the biocompatible polymeric matrix can be generally impermeable such that the water or moisture is present in an amount of about 0 wt. % based on the total weight of the biocompatible polymeric matrix, where the reduced permeability “deactivates” the biocompatible polymeric matrix so that a generally constant concentration of any therapeutic gases introduced to the matrix can be maintained until use. In other words, drying the biocompatible polymeric matrix to such low moisture levels reduces the permeability of the biocompatible polymeric matrix such that any therapeutic gases introduced to the matrix can remain at generally constant concentration levels until the biocompatible polymeric matrix is ready for use, at which time a separately-stored fluid or ambient moisture can be introduced to the matrix to “reactivate” it and increase its permeability to the therapeutic gas, thus facilitating delivery of the therapeutic gas to an area beneath a surface of intact skin or a wound site for treatment.

Although not required, the biocompatible polymeric matrix may further include a hydration control agent. The hydration control agent may be an isopropyl alcohol such as isopropanol; however, ethanol, glycerol, butanol, and/or propylene glycol and combinations thereof may also be used. If present, the hydration control agent can be present in an amount ranging from about 0.05 wt. % to about 5% wt. %, such as from about 0.1 wt. % to about 2.5 wt. %, such as from about 0.25 wt. % to about 1 wt. % based on the total weight of the biocompatible polymeric matrix of the present invention prior to drying. On the other hand, after dehydration, the hydration control agent is generally absent from the biocompatible polymeric matrix of the present invention.

Ammonium persulfate and tetramethylethylenediamine (TEMED) may also be included in the biocompatible polymeric matrix of the present invention. The ammonium persulfate can be a component of the biocompatible polymeric matrix of the present invention in an amount ranging from about 0.005 wt. % to about 0.5 wt. %, such as from about 0.01 wt. % to about 0.25% wt. %, such as from about 0.025 wt. % to about 0.1 wt. % based on the total weight of the biocompatible polymeric matrix of the present invention prior to drying. After dehydration, the ammonium persulfate can be present in an amount ranging from about 0.025 wt. % to about 5 wt. %, such as from about 0.05 wt. % to about 3% wt. %, such as from about 0.1 wt. % to about 1 wt. % based on the total weight of the biocompatible polymeric matrix of the present invention on a dry-weight basis. Additionally, the TEMED can be a component of the biocompatible polymeric matrix of the present invention in an amount ranging from about 0.001 wt. % to about 0.5 wt. %, such as about 0.01 wt. %

to about 0.25% w/w, such as about 0.025 wt. % to about 0.15 wt. % based on the total weight of the biocompatible polymeric matrix of the present invention prior to drying. On the other hand, after dehydration, the TEMED can be present in an amount ranging from about 0.01 wt. % to about 3 wt. %, such as from about 0.025 wt. % to about 2 wt. %, such as from about 0.05 wt. % to about 1 wt. % based on the total weight of the biocompatible polymeric matrix of the present invention on a dry-weight basis.

Further, one or more active agents can be incorporated into the biocompatible polymeric matrix 120 of the present invention. As shown in FIG. 1, the one or more active agents 110 can be present on at least a skin-contacting portion 180 of the matrix 120. For example, the active agent 110 can be incorporated throughout the matrix 120 as a result of inclusion in the matrix 120 during compounding. Alternatively and/or additionally, the active agent 110 can be applied to the matrix 120 utilizing dipping, coating, deposition, or spraying processes or other similar known techniques. In any event, the active agent can be transported or delivered to an area beneath a surface of intact skin or a wound site via the separately-stored fluid or ambient moisture from the skin or air, where the active agent moves along a diffusion gradient for delivery beneath the surface of intact skin or the wound site.

The active agents incorporated into the present invention are selected on the basis of the use of the system and matrix. Active agents and their effects are known by those skilled in the art and methods for including these agents into the matrices of the present invention are taught herein. The present invention contemplates the inclusion of one or more active agents, depending on the intended use.

If the system of the present invention is used for topical treatments, such as treatments for compromised tissues, the system can include active agents that aid in treatment of compromised tissues. For example, the system can be used for the treatment of wounds, in skin healing or for cosmetic applications. In such systems, the active agents can aid and improve the wound healing process.

Active agents include, but are not limited to, pharmaceuticals, biologics, chemotherapeutic agents, herbicides, growth inhibitors, anti-fungal agents, anti-bacterial agents, anti-viral agents, anti-fouling agents, anti-parasitic agents, mycoplasma treatments, deodorizing agents, growth factors, proteins, nucleic acids, angiogenic factors, anaesthetics, analgesics, mucopolysaccharides, metals, wound healing agents, growth promoters, indicators of change in the environment, enzymes, nutrients, vitamins, minerals, carbohydrates, fats, fatty acids, nucleosides, nucleotides, amino acids, sera, antibodies and fragments thereof, lectins, immune stimulants, immune suppressors, coagulation factors, neurochemicals, cellular receptors, antigens, adjuvants, radioactive materials, nutrients, nanoparticles, or therapeutic agents produced by a catalyst, any other agents that effect cells or cellular processes, or a combination thereof.

Examples of antimicrobial agents that can be used in the present invention include, but are not limited to, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

Growth factor agents that may be incorporated into the biocompatible polymeric matrix of the present invention include, but are not limited to, basic fibroblast growth factor (bFGF), acidic fibroblast growth factor (aFGF), nerve growth factor (NGF), epidermal growth factor (EGF), insulin-like growth factors 1 and 2, (IGF-1 and IGF-2), platelet derived growth factor (PDGF), tumor angiogenesis factor (TAF), vascular endothelial growth factor (VEGF), corticotropin releasing factor (CRF), transforming growth factors α and β (TGF-α and TGF-β), interleukin-8 (IL-8); granulocyte-macrophage colony stimulating factor (GM-CSF); the interleukins, and the interferons.

Other agents that may be incorporated into biocompatible polymeric matrix of the present invention are acid mucopolysaccharides including, but are not limited to, heparin, heparin sulfate, heparinoids, dermatitin sulfate, pentosan polysulfate, chondroitin sulfate, hyaluronic acid, cellulose, agarose, chitin, chitosan, dextran, carrageenan, linoleic acid, and allantoin.

Proteins that may be especially useful in the treatment of compromised tissues, such as wounds, include, but are not limited to, collagen, cross-linked collagen, fibronectin, laminin, elastin, and cross-linked elastin or combinations and fragments thereof. Adjuvants, or compositions that boost an immune response, may also be used in conjunction with the biocompatible polymeric matrix of the present invention.

Other wound healing agents that are contemplated in the present invention include, but are not limited to, metallic oxides and nanoparticles such as zinc oxide and silver ions, which have long been known to provide excellent treatment for wounds. Delivery of such agents, by the methods and compositions of the present invention, provide a new dimension of care for wounds.

It is to be understood that in preferred embodiments of the present invention, the active agents are incorporated into the matrix or system in such a manner so that the agents are released into the environment and/or retained within the matrix or system. In topical treatments, the agents are then delivered via transdermal or transmucosal pathways. The incorporated agents may be released over a period of time, and the rate of release can be controlled by the amount of cross-linking of the polymers of the matrices. In this way, the present invention retains its ability to affect the local environment, kill or inhibit microorganisms, boost the immune response, exert other alterations of physiological function and provide active agents over an extended period of time.

In another embodiment of the present invention, active agents can be incorporated directly into micro or macro-cavities of the matrix. The agents may be incorporated by absorption of agents by the matrix, and preferably by incorporation during the polymerization of the matrix. Regardless of the manner in which the active agent is incorporated into the biocompatible polymeric matrix of the present disclosure, the active agent can be present in an amount ranging from about 0.1 wt. % to about 25 wt. %, such as from about 0.25 wt. % to about 20 wt. %, such as from about 0.5 wt. % to about 10 wt. % based on the total weight of the biocompatible polymeric matrix of the present invention on a dry-weight basis.

The present invention also contemplates the use of one or more superabsorbent materials to facilitate the ability of the biocompatible polymeric matrix to absorb fluid, which, in turn, facilitates the delivery of a therapeutic gas such as oxygen to an area or intact skin or a wound site in an efficient manner. Suitable superabsorbent materials include hydrophilic polymers which, in crosslinked form, are capable of absorbing large quantities of fluids. In particular, hydrophilic polymers useful in this invention are those typically employed to make superabsorbent polymers (SAPs), such as water-absorbent polymers which contain carboxyl moieties. Among preferred carboxyl containing water-absorbent polymers are those derived from one or more ethylenically unsaturated carboxylic acids, ethylenically unsaturated carboxylic acid anhydrides or salts thereof. Additionally, the polymers can include comonomers known in the art for use in water-absorbent resin particles or for grafting onto the water-absorbent resins, including comonomers such as acrylamide, a vinyl pyrrolidone, a vinyl sulphonic acid or a salt thereof, acrylamidopropane sulfonic acid (AMPS), salts thereof, or phosphonic acid containing monomers, a cellulosic monomer, a modified cellulosic monomer, a polyvinyl alcohol, a starch hydrolyzate, the hydrolyzates of acrylamide copolymers, or crosslinked products of hydrolyzates of acrylamide copolymers. Examples of ethylenically unsaturated carboxylic acid and carboxylic acid anhydride monomers include, but are not limited to, acrylic acids, such as acrylic acid, methacrylic acid, ethacrylic acid, alpha-chloro acrylic acid, alpha-cyano acrylic acid, beta-methyl acrylic acid (crotonic acid), alpha-phenyl acrylic acid, beta-aryloyloxy propionic acid, sorbic acid, alpha-chloro sorbic acid, angelic acid, cinnamic acid, p-chloro cinnamic acid, beta-styd acrylic acid (1-carboxy-4-phenyl butadiene-1,3), itaconic acid, citraconic acid, mesaconic acid, glutaconic acid, maleic acid, fumaric acid and maleic acid anhydride. In some embodiments, the carboxyl containing water-absorbent polymers are derived from acrylic acid, methacrylic acid, or a salt thereof, with partially neutralized products of polyacrylic acids and crosslinked products of partially neutralized polyacrylic acids being especially preferred polymers. Mixtures of monomers can also be employed.

The biocompatible polymeric matrix contemplated by the present invention may take many physical forms, depending on the various uses of the matrix. The matrix may be left in place for a time period ranging from about 30 minutes to about 10 days, or any time period therebetween, and then removed. Further, the matrix can absorb at least 5 times its weight in fluid (e.g., water, saline, chemically simulated wound fluid, wound fluid, a wetting agent, or a combination thereof, etc.). For instance, the matrix, in some embodiments, can absorb from about 5 times to about 20 times its weight in fluid (e.g., water, saline, chemically simulated wound fluid, wound exudate, a wetting agent, or a combination thereof, etc.), such as from about 5 times to about 10 times its weight in fluid. In addition, the biocompatible polymeric matrix 120 of FIG. 1 can be molded into any suitable planar two-dimensional or non-planar three-dimensional shape (not shown), such that the biocompatible polymeric matrix of the present invention can conform to any part of the patient's body.

Regardless of its particular physical shape, the overall thickness of the biocompatible polymeric matrix can be selectively controlled to range from about 0.1 inches (2.54 millimeters) to about 1 inch (25.4 millimeters), such as from about 0.15 inches (3.81 millimeters) to about 0.99 inches (25.15 millimeters), such as from about 0.25 inches (6.35 millimeters) to about 0.95 inches (24.13 millimeters), such as from about 0.3 inches (7.62 millimeters) to about 0.9 inches (22.86 millimeters) such as from about 0.4 inches (10.16 millimeters) to about 0.8 inches (20.32 millimeters).

In addition, FIG. 2 shows one embodiment of a therapeutic gas preservation and delivery system 100 contemplated by the present invention. The system 100 includes a biocompatible polymeric matrix 120 that can be generally planar and can be made from one layer of material, where a skin contacting surface 180 can be placed on a generally flat or slightly curved area of skin. Further, although not shown, it is also to be understood that the system 100 can include two or more layers of material that include the biocompatible polymeric matrix 120. The system 100 also includes a separately-stored fluid 130 (e.g., water, saline, wetting agent, or combination thereof, etc.) that can be used to activate the biocompatible polymeric matrix 120 when it is in a deactivated state (e.g., exhibits reduced permeability or is generally impermeable to the therapeutic gas to maintain a constant concentration of the therapeutic gas prior to using the system 100 for therapeutic gas delivery, such as via drying or desiccation of the matrix 120). However, it is also to be understood that the present invention contemplates a biocompatible polymeric matrix 120 that can be activated via the addition or exposure of ambient moisture rather than by the addition of a separately-stored fluid. Accordingly, the term “separately-stored fluid” refers to a fluid such as a liquid, steam, saturated vapor or the like that is sequestered or isolated from the polymeric matrix and, when exposed or applied to the polymeric matrix, is configured to activate the polymeric matrix from an inactive or desiccated state to enable or enhance the delivery of therapeutic gas from the closed cells of the polymeric matrix. Such a “separately-stored fluid” may separately-stored by isolating the polymeric matrix itself (e.g., by containing or enveloping the polymeric matrix in a package) from the separated fluid for subsequent activation by exposing the inactive or desiccated polymeric matrix to the separated fluid (which may be in the form of steam or saturated vapor) to enable or enhance the delivery of therapeutic gas from the closed cells of the polymeric matrix or the “separately-stored fluid” may be separately-stored by packaging or isolating the fluid itself for subsequent activation by application of the fluid to the inactive or desiccated polymeric matrix to enable or enhance the delivery of therapeutic gas from the closed cells of the polymeric matrix.

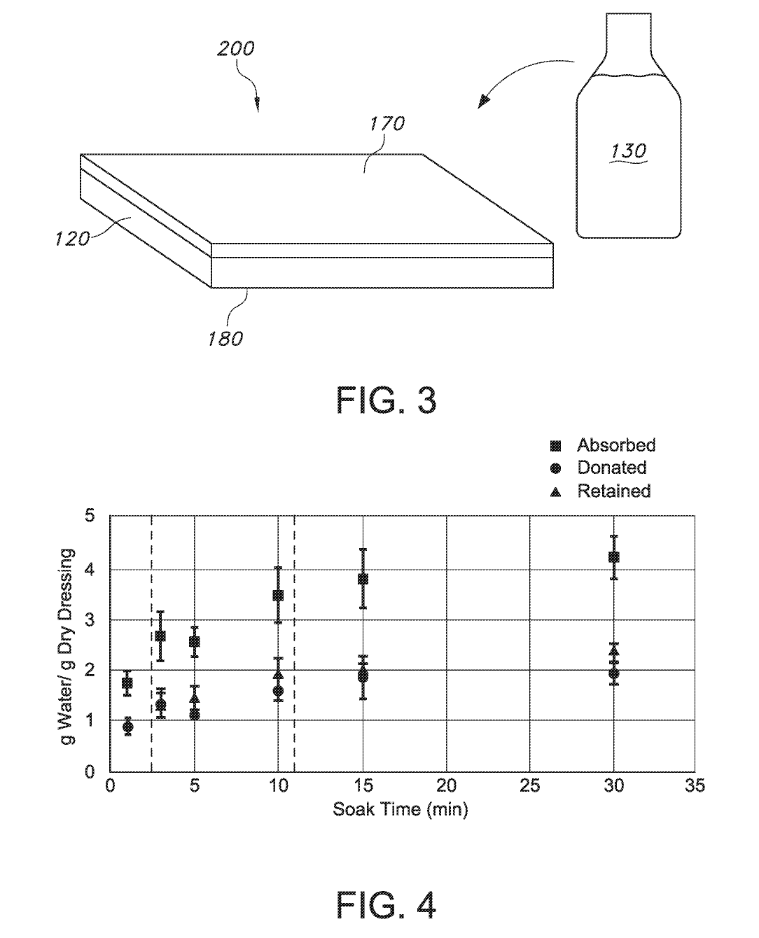

FIG. 3 shows another embodiment of a therapeutic gas preservation and delivery system 200 contemplated by the present invention. The system 200 includes a biocompatible polymeric matrix 120 that can be generally planar and can be made from one layer of material, where a skin contacting surface 180 can be placed on a generally flat or slightly curved area of skin. Further, although not shown, it is also to be understood that the system 100 can include two or more layers of material that include the biocompatible polymeric matrix 120. The system 200 also includes a separately-stored fluid 130 (e.g., water, saline, wetting agent, or combination thereof, etc.) that can be used to activate the biocompatible polymeric matrix 120 when it is in a deactivated state (e.g., exhibits reduced permeability or is generally impermeable to the therapeutic gas to maintain a constant concentration of the therapeutic gas prior to using the system 200 for therapeutic gas delivery, such as via drying or desiccation of the matrix 120). However, it is also to be understood that the present invention contemplates a biocompatible polymeric matrix 120 that can be activated via the addition of ambient moisture rather than by the addition of a separately-stored fluid. The system 200 can also include an occlusive backing 170 that prevents release of the therapeutic gas to the ambient environment and thus facilitates diffusion of the therapeutic gas (which has been dissolved in a fluid upon activation of the matrix) to an area beneath a surface of intact skin or a wound site.

The process for making the biocompatible polymeric matrix contemplated by the present invention can generally involve at least the steps of: providing a gelling mixture of at least one cross-linkable biocompatible polymer and an oxygen catalyst (e.g., a first reactant); pouring the gelling mixture into a suitable mold to create the desired planar two-dimensional or non-planar three-dimensional shape, cross-linking the biocompatible polymer of the gelling mixture to form a water swellable, cross-linked biocompatible polymer network; drying the gelling mixture to form a molded gel; adding a second reactant to the molded gel; and generating a plurality of closed cells (i.e., closed cells and walls) or a combination of closed cells and open cells containing oxygen in the molded gel by reacting the catalyst and the second reactant.

Generally speaking, the present invention contemplates a biocompatible polymeric matrix for preserving and delivering a therapeutic gas (e.g., oxygen). A feature of the biocompatible polymeric matrix is the formation of the foam or array of bubbles that entrap the gas. The foam or bubbles are formed by the permeation of the second reactant added to the formed matrix that includes a reactant. When the two reactants interact, a reaction occurs that liberates oxygen gas which becomes entrapped within the matrix. For example, a matrix has a carbonate catalyst (a reactant) incorporated within it. The formed matrix is then placed in the presence of the second reactant (e.g., a peroxide compound such as, for example, hydrogen peroxide). A catalytic decomposition of hydrogen peroxide occurs resulting in the liberation of oxygen gas which becomes entrapped as bubbles formed in situ. The hydrogen peroxide reactant is not part of the compounding of the matrix, but it is in the treatment after the formation of the matrix stock.

According to the process, a gelling mixture of at least one cross-linkable biocompatible polymer is prepared. This may be accomplished by creating a solution of a cross-linkable biocompatible polymer or a solution composed of a mixture of cross-linkable biocompatible polymers. The solution is desirably an aqueous solution or a solution where water component is the major component. According to the process, any of the active agents discussed above may be added to the gelling mixture. A catalyst for generating the gaseous oxygen (e.g., a first reactant) may be introduced into the gelling mixture. For example, the catalyst may be sodium carbonate, although any of the catalysts discussed above may be utilized. For instance, other catalysts such as other alkali and alkali earth compounds may be used provided they are consistent with the product being biocompatible. In addition, more than one catalyst may be used. For example, one catalyst may be derived from a group consisting of salts of alkali metals and alkali earth metals and the second catalyst may include, but are not limited to, organic and inorganic chemicals such as cupric chloride, ferric chloride, manganese oxide, sodium iodide and their equivalents. Other catalysts, include, but are not limited to enzymes such as lactoperoxidase and catalase. Desirably, the catalyst is a material that interacts with the second reactant.

The polysaccharide, plasticizer and/or a hydration control agent may then be added to the gelling mixture.

The cross-linkable biocompatible polymer of the gelling mixture is then cross-linked to form a water swellable, cross-linked biocompatible polymer network. This may be accomplished by activating a cross-linking agent already present in the gelling mixture, adding or applying a cross-linking agent to the gelling mixture, dehydrating or removing solvent from the gelling mixture, and/or otherwise creating conditions in the gelling mixture that causes cross-linking (e.g., pH, heat, various forms of radiation including electromagnetic radiation and x-rays, ultrasonic energy, microwave energy and the like).

For example, the gelling mixture may be cross-linked by activating a cross-linking agent and dehydrating the gelling mixture in the mold, resulting in a molded gel that can be readily handled. As another example, the gelling mixture may be cross-linked by placing the mold in a conventional oven at an elevated temperature (e.g., 45 to 65° C.) and dehydrating for 2 to 20 hours until the gelling mixture reaches a consistency similar to a leathery gum. Desirably, the resulting molded gel is flexible and elastic, and may be a semi-solid scaffold that is permeable to substances such as aqueous fluids, silver salts, and dissolved gaseous agents including oxygen. Though not wishing to be bound by any particular theory, it is thought that the substances permeate the matrix through movement via intermolecular spaces among the cross-linked polymer.

A second reactant is then added to the molded gel. This may be accomplished by pouring a solution of the second reactant over the molded gel, or by spraying, brushing, coating or applying the second reactant. In one particular embodiment, the second reactant is hydrogen peroxide. The hydrogen peroxide may be used at a concentration ranging from about 1 wt. % to about 30 wt. % or more, such as from about 3 wt. % to about 20 wt. %. Other peroxides may be substituted, including, but not limited to, urea peroxide, sodium peroxide or other peroxy compounds of alkali metal or alkali earth metals. The second reactant preferably is present in aqueous solution or a solution wherein water is major component.

The second reactant is absorbed into the molded gel and permeates the swellable, (e.g., water-swellable) cross-linked biocompatible polymeric matrix. The degree of swelling may be increased by adding a polysaccharide such as dextrin, guar gum, xanthan gum, locust bean gum, carrageenan, sodium alginate, methyl cellulose, hydroxypropyl cellulose, carboxymethyl cellulose, etc. as well as by adding a hydration control agent such as glycerol. By making the cross-linked biocompatible polymer network sufficiently water-swellable, the second reactant (e.g., hydrogen peroxide) may be adequately absorbed into the cross-linked biocompatible polymer network.

According to this process, when the second reactant is absorbed and permeates into the cross-linked biocompatible polymeric matrix, a gas is generated when the catalyst (i.e., the first reactant) reacts with the second reactant. The resulting gas can be oxygen gas or any other suitable therapeutic gas. The matrix of the present invention forms a foam or array of bubbles that entrap the gas. That is, a plurality of closed cells containing oxygen is generated in the molded gel by reaction between the catalyst (i.e., the first reactant) and the second reactant.

For example, a cross-linked biocompatible polymeric matrix may have a carbonate catalyst (i.e., a first reactant) incorporated within it. The cross-linked biocompatible polymeric matrix is then placed in the presence of the second reactant, hydrogen peroxide. A catalytic decomposition of hydrogen peroxide occurs resulting in the liberation of oxygen gas which becomes entrapped as bubbles formed in situ. The hydrogen peroxide reactant is not part of the compounding of the matrix, but it is added after the formation of the matrix stock.

In finished form, a planar two-dimensional or non-planar three-dimensional matrix is formed that can have any shaped determined by the mold in which the solution is placed.

It is contemplated that the process of the present invention may further include the step of heating or adding energy to the molded gel and second reactant to speed up the reaction.

Once the biocompatible polymeric matrix is formed and modified (e.g., by drying, desiccation, etc.) so that it has the desired reduced permeability to a therapeutic gas such as oxygen, the matrix can be stored along with a separate fluid until it is needed for the treatment of an open wound or an area of intact skin that is hypoxic. In use, the matrix is soaked in the separate fluid for a time period ranging from about 5 seconds to about 30 minutes, such as from about 30 seconds to about 25 minutes, such as from about 1 minute to about 20 minutes, such as from about 3 minutes to about 15 minutes and applied to the wound site or surface of intact skin so that a surface of the biocompatible polymeric matrix is in contact with the wound site or intact skin. Although the entire matrix can be immersed in the fluid, in some embodiments, introducing fluid to an outer-facing surface of the matrix, such as the surface on which an optional occlusive backing may be positioned, can be avoided in order to reduce slipperiness when positioning the matrix in its desired location. For instance, it may be desirable to only immerse about 60% to about 80%, such as about 70% to about 75% of the thickness of the matrix, including the skin-contacting portion, in the fluid. Once positioned over the surface of intact skin or wound site, the matrix can be left in place for up to about 10 days, during which time the therapeutic gas can be delivered to an area beneath the surface of skin or wound site or transported through the surface of intact skin or transported to the wound site.

The present invention may be better understood with reference to the following examples.

### EXAMPLE 1

### Matrix Formation

A biocompatible polymeric matrix containing oxygen gas was produced to test its ability to deliver oxygen after being subjected to various conditions as described in the following Examples. The matrix was a planar, square shaped wound dressing, as shown in FIGS. 1 and 2, with oxygen gas contained in the cells of the matrix. The closed cell foam composition/solution for use in forming the biocompatible polymeric matrix was generally made in accordance with the process described in the Detailed Description above as well as U.S. Pat. No. 7,160,553 issued Jan. 9, 2007 and U.S. Pat. No. 8,679,523 issued Mar. 25, 2014 to Gibbins, et al. The various components used in forming the biocompatible polymeric matrix (containing oxygen) and their respective weight percentages are shown below in Tables 1 and 2:

TABLE 1
Intermediate Gel Matrix
Intermediate gel matrix

| Ingredient | % by wt. |
|---|---|
| Water | 86.46 |
| Acrylamide | 5.74 |
| Bis Acrylamide | 0.07 |
| Guar Gum | 0.57 |
| Isopropyl Alcohol | 1.03 |
| Ammonium Persulfate | 0.06 |
| TEMED | 0.08 |
| Glycerol | 5.81 |
| Sodium Carbonate | 0.18 |
| Total | 100 |

TABLE 2
Peroxide Solution
Peroxide solution

| Ingredient | % by wt. |
|---|---|
| Hydrogen Peroxide | 66.60 |
| Water | 33.30 |
| Tween 20 | 0.10 |
| Total | 100 |

To form the matrices used in the following examples, the components from Table 1 were added to a batch tank and mixed into a gel solution. The gel solution was then poured from the batch tank into molds and allowed to polymerize. The polymerized gel sheets were then removed from the molds and dried to remove water/moisture. The dried gel sheets were then dipped into the peroxide solution of Table 2 and placed into an oven to begin the foaming process and incorporate the oxygen gas into the matrix. After the foaming process was complete, the oxygen-filed foam was cut to size and dried again to remove moisture. Once the moisture was removed, the foam was impermeable to gas transfer (i.e., the foam was in a deactivated state) and was then packaged in a gas/moisture impermeable pouch. Then, the foam was sterilized.

### EXAMPLE 2

The biocompatible polymeric matrix formed in Example 1 was soaked in water on one side for times periods of one minute, three minutes, five minutes, ten minutes, fifteen minutes, and thirty minutes, and the moisture (water) absorption, retention, and donation levels based on grams of water per grams of the dry matrix were determined. As shown in FIG. 4, the biocompatible polymeric matrix was able to donate or supply moisture after being soaked in water for a time period ranging from one minute to thirty minutes. Generally, about half of the moisture (water) was donated and about half of the moisture (water) was retained in the dressing until the matrix's ability to absorb, retain, and donate moisture leveled off at around the fifteen minute soak time mark. Further, FIG. 4 shows that increasing the soak time increased the amount of moisture that could be donated, with soak times of three minutes to ten minutes providing significant moisture donation.

### EXAMPLE 3

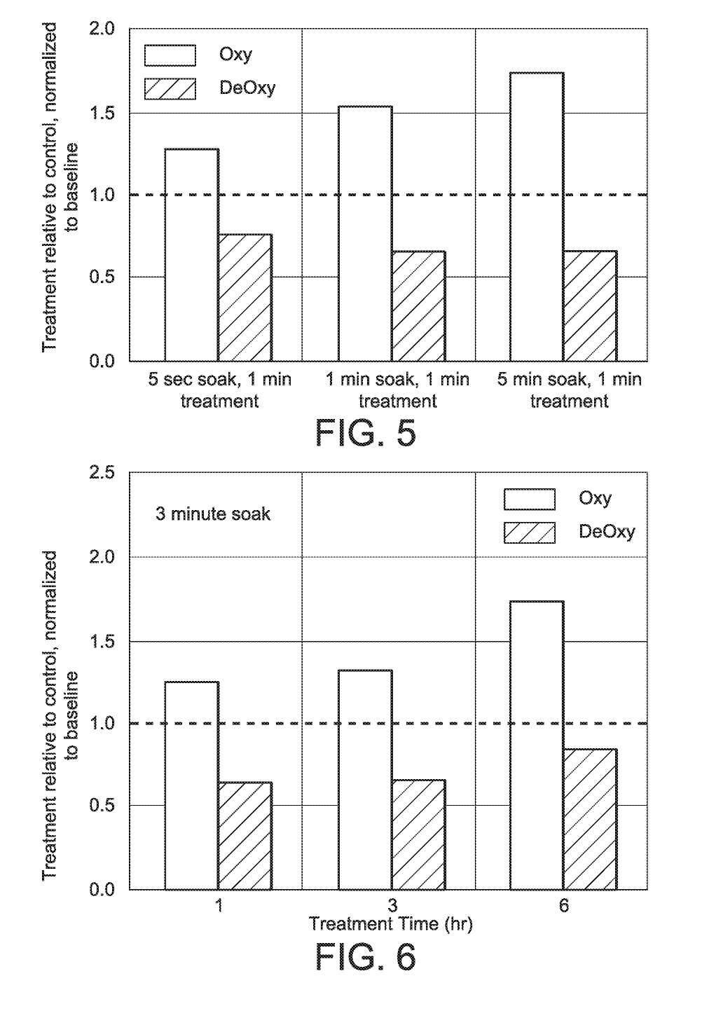

The biocompatible polymeric matrix formed in Example 1 was soaked in water for five seconds, one minute, and five minutes and was then applied to a surface of intact skin along with a dry control matrix. The amount of dissolved oxygen diffusing from the matrix through the surface of intact skin via the water as a carrier fluid after about 1 minute of contact was measured by determining the levels of oxyhemoglobin (hemoglobin with 1-4 bound oxygen molecules) and deoxyhemoglobin (hemoglobin with 0 bound oxygen molecules) in a capillary bed 2 millimeters below the surface of intact skin via hyperspectral imaging (OxyVu-2 from HyperMed, Inc.). As shown in FIG. 5, soaking the matrix in water for a time period ranging from five seconds to five minutes increased the amount of oxygen that was able to penetrate intact skin compared to a dry control matrix. Specifically, soaking the matrix in water increased the amount of oxygen delivered by a factor of about 1.25 for a one minute soak, by a factor of about 1.5 for a one minute soak, and by a factor of about 1.75 for a five minute soak. Further, as shown, soaking the matrix in water for five minutes provided for the largest increase in oxygen penetration compared to the dry control matrix.

### EXAMPLE 4

The biocompatible polymeric matrix formed in Example 1 was soaked in water for three minutes, five minutes, and fifteen minutes and was then applied to a surface of intact skin along with a dry control matrix. The amount of dissolved oxygen diffusing from the matrix through the surface of intact skin via the water as a carrier fluid after about one hour, three hours, six hours, twelve hours (five minute soak only), eighteen hours (five minute soak only) and twenty-four hours (five minute soak only) of treatment/contact was measured by determining the levels of oxyhemoglobin (hemoglobin with 1-4 bound oxygen molecules) and deoxyhemoglobin (hemoglobin with 0 bound oxygen molecules) in a capillary bed 2 millimeters below the surface of intact skin via hyperspectral imaging (OxyVu-2 from HyperMed, Inc.).

As shown in FIG. 6, soaking the matrix in water for three minutes increased the amount of oxygen that was able to penetrate intact skin compared to a dry control matrix through at least six hours of treatment. Specifically, soaking the matrix in water for three minutes increased the amount of oxygen delivered by a factor of about 1.25 for a one hour treatment, by a factor of about 1.35 for a three hour treatment, and by a factor of about 1.75 for a six hour treatment.

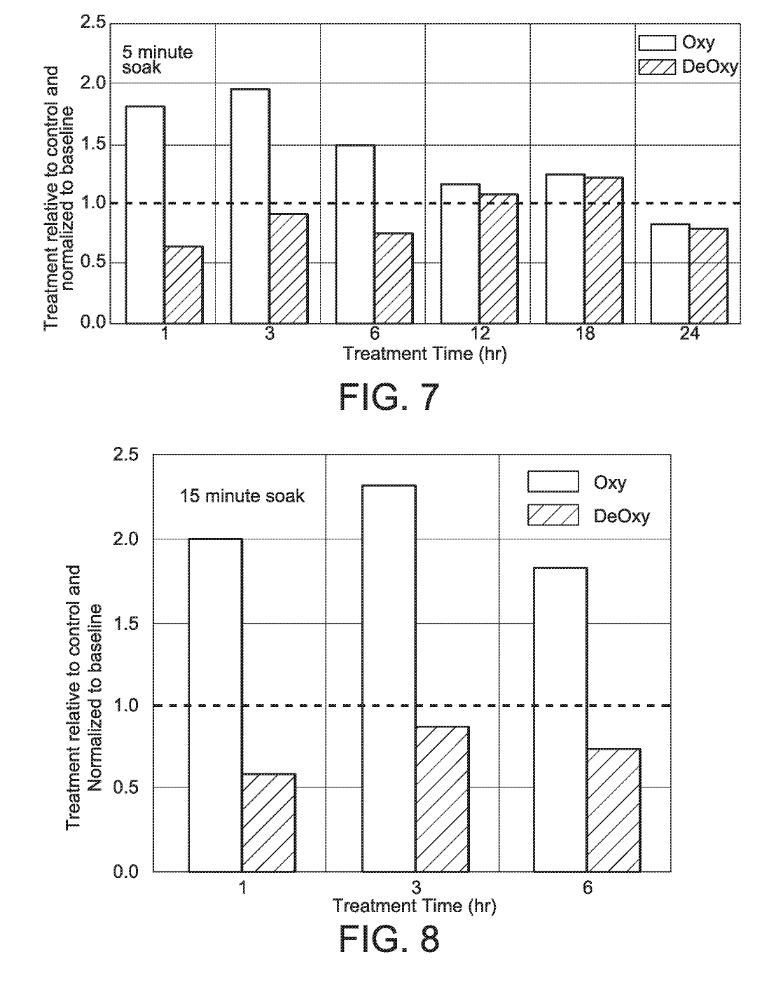

As shown in FIG. 7, soaking the matrix in water for five minutes increased the amount of oxygen that was able to penetrate intact skin compared to a dry control matrix up until about eighteen hours of treatment. Specifically, soaking the matrix in water for five minutes increased the amount of oxygen delivered by a factor of about 1.75 for a one hour treatment, by a factor of about 2 for a three hour treatment, by a factor of about 1.5 for a six hour treatment, by a factor of about 1.2 for a twelve hour treatment, and by a factor of about 1.2 for an eighteen hour treatment.

As shown in FIG. 8, soaking the matrix in water for fifteen minutes increased the amount of oxygen that was able to penetrate intact skin compared to a dry control matrix through at least six hours of treatment. Specifically, soaking the matrix in water for fifteen minutes increased the amount of oxygen delivered by a factor of about 2 for a one hour treatment, by a factor of about 2.25 for a three hour treatment, and by a factor of about 1.75 for a six hour treatment. Thus, the fifteen minute soak provided the largest increase in oxygen delivery as compared to the dry control matrix as compared to the three minute soak and the five minute soak.

### EXAMPLE 5

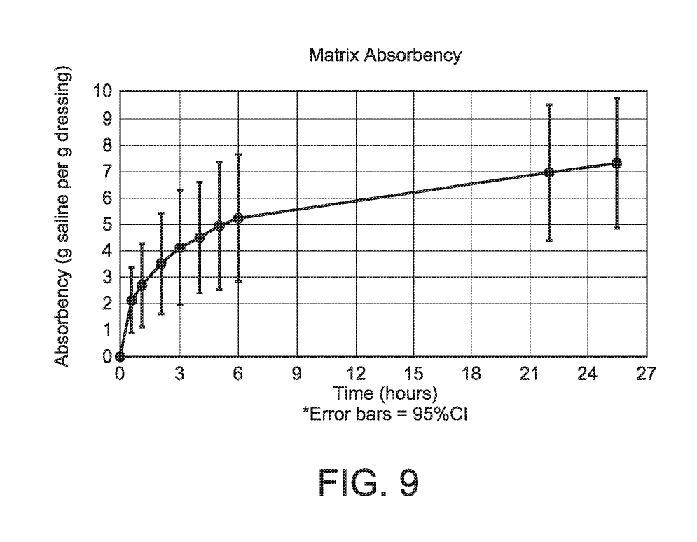

The ability of the biocompatible polymeric matrix formed in Example 1 to absorb saline was demonstrated. As shown in FIG. 9, the matrix was able to increase the amount of saline absorbed over a time period of 0 hours to 26 hours, where at 26 hours, the matrix had absorbed just over 7 grams of saline per gram of matrix. Although not shown graphically, the matrix was generally able to absorb twice as much water per gram as compared to the saline.

### EXAMPLE 6

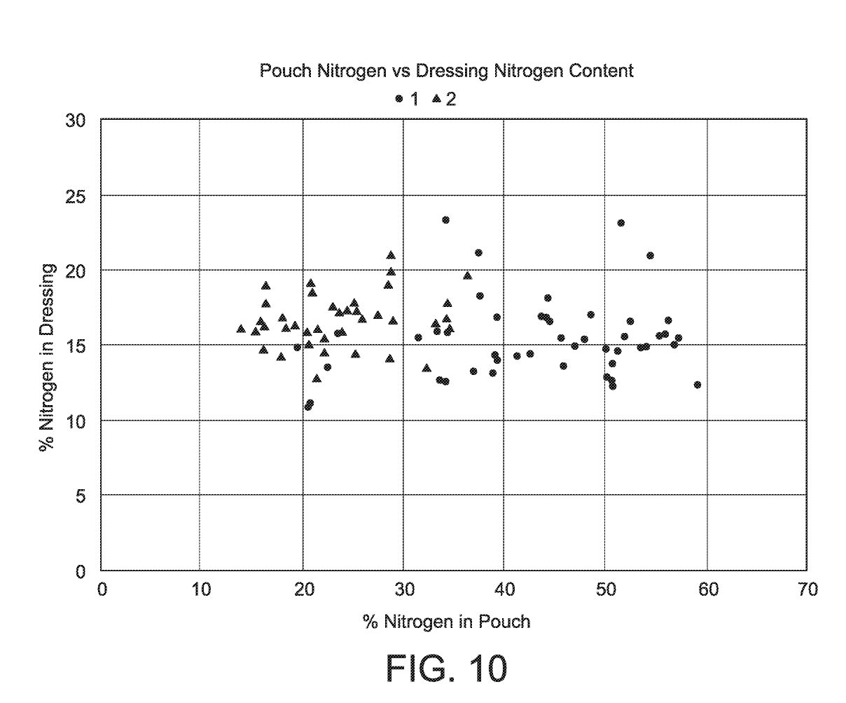

The biocompatible polymeric matrix formed in Example 1 was sealed in a pouch containing varying concentrations of oxygen and nitrogen, and samples were aged for time periods ranging from 2 months to 20 months to test the stability of the gas concentration of the matrix in a dry environment. As shown in FIG. 10, the nitrogen gas composition in the matrix did not change as the amount of nitrogen gas in the pouch was increased for two lots of samples, showing that the matrix was in a generally impermeable or deactivated state because it was able to maintain a generally constant concentration of gas within the matrix despite increases in ambient gas concentration.

### EXAMPLE 7

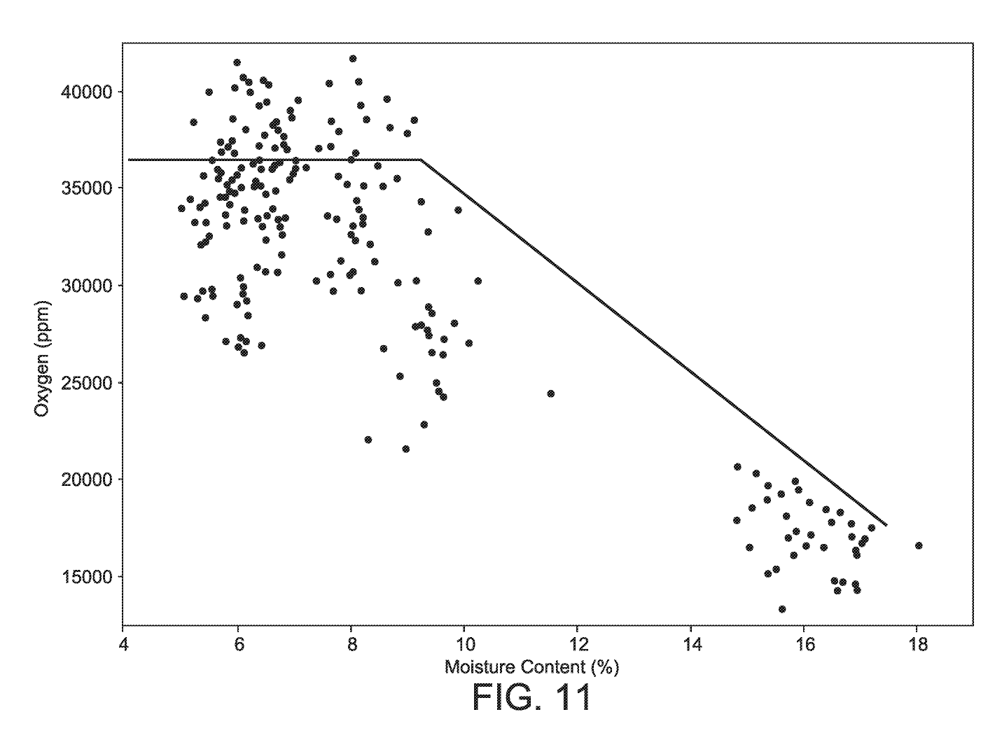

Samples of the biocompatible polymeric matrix formed in Example 1 were provided, where the samples had a moisture content ranging from about 5% to about 18%. The oxygen content in each of the samples was determined in parts per million (ppm). As shown in FIG. 11, increasing the moisture content of the matrix, and thus its permeability to oxygen, decreased the amount of oxygen present in the matrix, where the matrix samples having a moisture content ranging from about 5% to about 9.2% exhibited increased levels of oxygen and thus less diffusion of the oxygen out of the matrix, indicating that the matrices having moisture levels below about 9.2% were less permeable to the oxygen than matrices having a moisture content greater than about 9.2%.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A therapeutic gas preservation and delivery system comprising: (1) a biocompatible polymeric matrix including a polymer and closed cells, wherein a generally constant concentration of a therapeutic gas is stored within biocompatible polymeric matrix, further wherein the biocompatible polymeric matrix includes a surface configured to directly contact a surface of intact skin or a wound site; and (2) a separately-stored fluid, wherein maintaining separation between the biocompatible polymeric matrix and the separately-stored fluid preserves the concentration of the therapeutic gas stored in the biocompatible polymeric matrix, further wherein adding or exposing the separately-stored fluid to the biocompatible polymeric matrix facilitates delivery of the therapeutic gas to an area beneath the surface of intact skin or the wound site, wherein the therapeutic gas is dissolved in the separately-stored fluid that has been added or exposed to the biocompatible polymeric matrix.

2. The therapeutic gas preservation and delivery system of claim 1, wherein the biocompatible polymeric matrix is exposed to the separately-stored fluid for a time period ranging from about 5 seconds to about 30 minutes.

3. The therapeutic gas preservation and delivery system of claim 1, wherein adding or exposing the separately-stored fluid to the biocompatible polymeric matrix results in an on-demand therapeutic gas delivery rate ranging from about 1 milliliter/(m²·hour) to about 10,000 milliliters/(m²·hour).

4. The therapeutic gas preservation and delivery system of claim 1, wherein the separately-stored fluid comprises water, saline, a wetting agent, chemically simulated wound fluid, wound exudate, or a combination thereof.

5. The therapeutic gas preservation and delivery system of claim 1, wherein the therapeutic gas is dissolved in the separately-stored fluid after the separately-stored fluid is added or exposed to the biocompatible polymeric matrix such that the therapeutic gas moves along a diffusion gradient for delivery beneath the surface of intact skin or the wound site.

6. The therapeutic gas preservation and delivery system of claim 1, wherein the therapeutic gas comprises oxygen, nitrogen, nitric oxide, carbon dioxide, air, nitrous oxide, hydrogen sulfide, a gaseous prostaglandin, a flurane derivative, or a combination thereof.

7. The therapeutic gas preservation and delivery system of claim 1, wherein the therapeutic gas is transported through the surface of intact skin or transported to the wound site for up to about 10 days.

8. The therapeutic gas preservation and delivery system of claim 1, wherein the biocompatible polymeric matrix is capable of absorbing at least five times its weight in fluid.

9. The therapeutic gas preservation and delivery system of claim 1, wherein the biocompatible polymeric matrix is generally free of moisture prior to addition of the separately-stored fluid.

10. The therapeutic gas preservation and delivery system of claim 1, wherein the biocompatible polymeric matrix exhibits a low permeability or impermeability to the therapeutic gas prior to addition of the separately-stored fluid and exhibits an increased permeability to the therapeutic gas after addition of the separately-stored fluid.

11. The therapeutic gas preservation and delivery system of claim 1, wherein the biocompatible polymeric matrix is hydrophilic.

12. The therapeutic gas preservation and delivery system of claim 1, wherein the polymer is at least partially cross-linked.

13. The therapeutic gas preservation and delivery system of claim 1, wherein the polymer is an absorbent, elastomeric, and biocompatible polymer or copolymer.

14. The therapeutic gas preservation and delivery system of claim 13, wherein the absorbent, elastomeric, and biocompatible polymer or copolymer comprises polyacrylamide, polyacrylate, polyhydroxyethyl methacrylate, polymethacrylamide, polyester, polyethylene glycol, polyether, polyurethane, polyethylene oxide, polyvinyl pyrrolidone, a silicone elastomer, or a combination thereof.

15. The therapeutic gas preservation and delivery system of claim 13, wherein the biocompatible polymeric matrix further comprises a polysaccharide.

16. The therapeutic gas preservation and delivery system of claim 1, wherein the biocompatible polymeric matrix further comprises an active agent.

17. The therapeutic gas preservation and delivery system of claim 16, wherein adding or exposing the separately-stored fluid to the biocompatible polymeric matrix facilitates delivery of the active agent to an area beneath the surface of intact skin or the wound site, wherein the active agent is dissolved in the separately-stored fluid that has been added or exposed to the biocompatible polymeric matrix.

18. The therapeutic gas preservation and delivery system of claim 1, wherein the biocompatible polymeric matrix further comprises a superabsorbent material.

19. The therapeutic gas preservation and delivery system of claim 1, comprising one or more layers of the biocompatible polymeric matrix.

20. The therapeutic gas preservation and delivery system of claim 1, wherein the system includes an occlusive backing layer disposed on a surface of the biocompatible polymeric matrix, wherein the occlusive backing layer is exposed to the ambient environment.

21. The therapeutic gas preservation and delivery system of claim 1, wherein the biocompatible polymeric matrix further comprises open cells.

22-67. (canceled)
